(19) United States
(12) Patent Application Publication
(10) Pub. No.: US 2009/0112307 A1
(43) Pub. Date: Apr. 30, 2009

(54) STENT HAVING A BASE BODY OF A BIOINERT METALLIC IMPLANT MATERIAL

(75) Inventors: Claus Harder, Uttenreuth (DE); Alexander Borck, Aurachtal (DE); Gerd Bayer, Erlangen (DE); Matthias Fringes, Ansbach (DE)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(73) Assignee: BIOTRONIK VI PATENT AG, Baar (CH)

(21) Appl. No.: 12/255,852

(22) Filed: Oct. 22, 2008

(30) Foreign Application Priority Data
Oct. 24, 2007 (DE) 10 2007 050 668.8

(51) Int. Cl. A61F 2/44

(52) U.S. Cl. 623/1.15; 623/1.44

(57) ABSTRACT

A stent having a base body of a bioinert metallic implant material wherein the base body is covered with an SiC coating. An external surface of the base body has a plurality of cavities which are filled with an antiproliferative active ingredient, A luminal surface of the base body has a coating which contains or comprises attractors for endothelial cells.

### PRIORITY CLAIM

This patent application claims priority to German Patent Application No. 10 2007 050 668.8, filed Oct. 24, 2007, the disclosure of which is incorporated herein by reference in its entirety.

### FIELD

The present disclosure relates to a stent having a base body of a bioinert metallic implant material.

### BACKGROUND

Implantation of stents has become established as one of the most effective therapeutic measures for treatment of vascular diseases. Stents assume a supporting function in the hollow organs of a patient. Stents of a traditional design have a tubular base body with a filigree supporting structure of metallic struts, initially in a compressed form for introduction into the patient's body and then widened at the site of use. One of the main areas for use of such stents is for permanently or temporarily dilating vascular obstructions, in particular, constrictions (stenoses) of the coronary vessels. In addition, aneurysm stents are also known, serving to support damaged vascular walls.

The basic body of the stent comprises an implant material. For purposes of the present disclosure, an implant material is a nonviable material that is used in medicine and interacts with biological systems. The basic prerequisites for use of a material as an implant material which is in contact with the body's physical environment when used as intended is its physical compatibility (biocompatibility). For purposes of the present disclosure, biocompatibility refers to the ability of a material to induce an appropriate tissue reaction in a specific application. This includes adaptation of the chemical, physical, biological and morphological surface properties of an implant to the recipient tissue with the goal of a clinically desired interaction. The biocompatibility of the implant material also depends on the chronological course of the reaction of the biosystem in which it is implanted. Thus irritation and inflammation occur relatively briefly and can lead to tissue changes. Biological systems thus react in different ways, depending on the properties of the implant material. According to the reaction of the biosystem, the implant materials can be subdivided into bioactive, bioinert and degradable/absorbable materials. For the purposes of the present disclosure, only bioinert or more specifically permanent metallic implant materials are of interest for stents.

A biological reaction to metallic elements depends on the concentration, duration of exposure and how the metallic elements are administered. Frequently, simply the presence of an implant material leads to inflammation reactions that may be triggered by mechanical stimuli, chemical substances or even metabolites. The inflammation process is usually accompanied by migration of neutrophilic granulocytes and monocytes through the vascular walls and migration of lymphocyte effector cells, forming specific antibodies to the inflammation stimulus, activation of the complement system and the release of complement factors which act as mediators and ultimately the activation of blood coagulation. An immunological reaction is usually closely associated with the inflammation reaction and may lead to sensitization and allergization. Instant restenosis due to excessive neointimal growth, which is caused by a great proliferation of arterial smooth muscle cells and a chronic inflammation reaction, is a significant problem with stent implantation into blood vessels.

It is known that a higher measure of biocompatibility and thus an improvement in restenosis rate can be achieved if metallic implant materials are provided with coatings of materials having a particularly high tissue compatibility. These materials are usually of an organic or synthetic polymer nature and to some extent of a natural origin.

Another strategy for preventing restenosis concentrates on inhibiting proliferation through medication. Active ingredient-coated stents (also known as drug eluting stents (DES)) are known and are very potent in suppressing proliferation of smooth human vascular muscle cells. Examples include stents coated with the active ingredient sirolimus or paclitaxel.

It is a disadvantage of the known DES that due to the nonspecific mechanism of action of the antiproliferative substances used so far, endothelialization is hindered or suppressed [see M. Joner, A. V. Finn, A. Farb, E. K. Mont, F. D. Kolodgie, E. Ladich, R. Kutys, K. Skorija, H. K. Gold, and R. Virmani. Pathology of drug-eluting stents in humans: delayed healing and late thrombotic risk. J. Am. Coll. Cardiol. 48 (1):193-202, 2006]. Inadequate endothelialization leads to a higher risk of occurrence of in-stent thromboses which, in the opinion of clinical experts, have a fatal outcome in approximately half of all cases.

Furthermore, the known DES has the disadvantage that growth into the vascular walls proceeds with a delay [see A. V. Finn, G. Nakazawa, M. Joner, F. D. Kolodgie, E. K. Mont, H. K. Gold, and R. Virmani. Vascular responses to drug eluting stents: importance of delayed healing. Arterioscler. Thromb. Vasc. Biol. 27 (7):1500-1510, 2007]. The antiproliferative effect of the conventional DES substances today almost completely suppresses the development of a neointima. Development of a restenosis is, therefore, successfully inhibited; but growth of the stent into the vascular wall is also inhibited. It is often observed here that the implanted stent is no longer in contact with the vascular wall, at least to some extent. Flow turbulences with development of a thrombus and bulges (aneurysms) in the vascular wall may develop and, in the worst case, may even rupture.

Finally, most known DES use permanent polymers as active ingredient reservoirs. These polymers have a low biocompatibility and some of the polymers may also be responsible for the occurrence of late thromboses.

Due to these disadvantages, the risk of an increased incidence of subsequent in-stent thromboses, which are fatal in approximately half of all cases, is increased in comparison with that observed with all-metal stents.

Despite the progress achieved, further improvement in the integration of stents into the biological environment and, therefore, a reduction in the incidence of restenoses would be desirable.

### SUMMARY

The present disclosure describes several exemplary embodiments of the present invention.

One aspect of the present disclosure provides a stent having a base body of a bioinert metallic implant material, the stent comprising a) a base body at least partially covered with an SiC coating; b) an external surface of the base body having a plurality of cavities which are at least partially filled with an antiproliferative active ingredient; and c) a luminal surface of the base body having a coating comprising attractors for endothelial cells.

### BRIEF DESCRIPTION OF THE DRAWINGS

Various aspects of the present disclosure are described hereinbelow with reference to the accompanying figure.

FIG. 1 shows a schematized section through a strut of the supporting structure of one exemplary embodiment of a stent according to the present disclosure.

### DETAILED DESCRIPTION

The present disclosure is based on the finding that a combination of an SiC coating of the base body, an antiproliferative active ingredient introduced into a plurality of cavities on the outside of the stent, and a coating containing an attractor for endothelial cells on the luminal side of the base body leads to a stent having optimal properties for the intended application. Previous approaches in stent optimization with stents coated with an active ingredient have been directed primarily at the choice of active ingredient. Other factors, such as the geometric design and mechanical parameters of the base structure, play practically no role.

CoCr, 316L or nitinol (nickel titanium) is preferred as the implant material for the base body. The stent may be in the form of a filigree supporting structure, preferably with a helical design such as that known from European Patent Application No. 1 430 854 A, for example.

The base body of the stent is covered with a semiconducting, insulating and passivating SiC layer. In long-term experiments, it has been found that SiC-coated implants are covered with new growth of endothelial cells and have a surface that is much more hydrophilic than that of traditional stents. In this way, the adhesion of the active ingredients/attractors applied to the luminal and mural sides can also be increased.

The mural surface (outer surface) of the stent has a plurality of cavities. These recesses in the outer surface of the stent have dimensions of a few micrometers and can be produced by laser methods, for example. The introduction of active ingredients into cavities offers the advantage that a coating which serves as a matrix for embedding the active ingredients may be omitted. The components of such matrices or their degradation products are often the cause of intolerance reactions, which may become the starting point for restenotic processes. This pertains, in particular, to most of the polymer matrices used in practice. One skilled in the art will be able to easily ascertain the exact number, position, contour and design of these cavities on the basis of the stent design available and the quantity of active ingredient to be introduced. Blind holes according to the design disclosed in International Patent Publication No. WO 2006/133223 are preferred.

The antiproliferative substance introduced into the cavities is eluted only into the vascular wall after implantation and is largely kept away from the luminal surface. The substance, therefore, has practically no inhibiting effect on endothelialization. The antiproliferative active ingredient is preferably selected from the taxols and taxans, preferably from the group consisting of paclitaxel, sirolimus, and especially derivatives of rapamycin, such as everolimus or biolomus. Sirolimus and the derivatives of rapamycin have delayed-release kinetics in comparison with other active ingredients applied to stents and thus ensure that the active ingredient may also be used even without a matrix/coating that serves to delay its release.

In addition, the SiC layer on the luminal side promotes adhesion of the active ingredient and subsequently promotes the growth into the vascular wall and suppresses negative effects due to its high biocompatibility.

The luminal surface of the stent is also covered with the SiC layer, which has been proven to promote rapid endothelialization. The luminal surface of the base body is preferably contoured to increase the surface area, i.e., the luminal surface of the base body has a texture. The texture of the luminal surface should preferably be designed so that attachment of endothelial cells is promoted. These textures can be created by roughening the surface. This is accomplished by sputtering, lasering or passivating, for example. A texture produced by sputtering, which is coated with the SiC layer in the same process, is especially preferred.

Finally, there is a coating on the luminal side which contains or comprises attractors with endothelium-specific binding possibilities. Chemical units bound covalently to the surface are considered to be attractors in this context, allowing the preferred binding of endothelial cells. These attractors include, in particular, antibodies (e.g., CD133), aptamers or RGD peptides, in particular cyclic RGDs (cRGD).

In this way, rapid and reliable endothelialization is achieved, also having an antiproliferative effect through its physiological functionality, e.g., emission of NO. Coated stents at the present time (e.g., coated with sirolimus or paclitaxel) prevent or at least interfere with endothelialization on the luminal side. Therefore, concomitant medication therapy for prevention of thrombosis for a period of many years is necessary. This disadvantage can be overcome with the help of the stent of the present disclosure. After the medication has been completely released and after endothelialization there remains only a permanent stent coated with SiC in the blood vessel. SiC has proven to have a high degree of biocompatibility for more than ten years.

Due to the introduction of the active ingredients into cavities on the mural side, an extensive polymer-free stent coating can be achieved. The active ingredient may be introduced into the cavities in a targeted but known manner. For example, it is also conceivable to encapsulate the active ingredients according to the usual galenical formulations, e.g., to achieve a time delay in the release of active ingredients. Galenics is a subfield of pharmaceutical technology concerned with the form of administration of medication. Traditionally, an object of galenics was to protect sensitive active ingredients from gastric acid. This was achieved by using acid-stable polymers, such as Eurdagit™ (available from Rohm Pharma), which are stable in acid but are susceptible to bases, to coat the tablets. It has been found that this fundamental idea is also of interest for stents because active ingredients, such as rapamycin, should be released over a long period of time. Galenics is helpful, in particular, when the cavities filled with the active ingredient are to be galenically “encapsulated” (e.g., with Eurdagit™) as in the present disclosure.

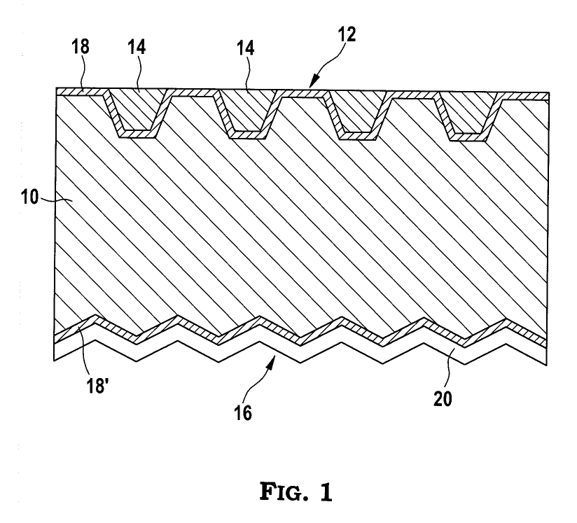

In FIG. 1, a base body 10 comprising a permanent metallic implant material (e.g., CoCr) has a plurality of cavities 14 in its mural surface 12, these cavities being producible by conventional processing methods (e.g., by means of a laser beam). The section shown in FIG. 1 runs through a few of the cavities 14 on the mural surface 12. The dimensions of the cavities 14 are in the range of a few micrometers.

A luminal surface 16 of the base body 10 is also textured. As shown in FIG. 1, as an example, the surface has a wavy contour which leads to an increase in the surface area of the luminal surface 16 and accordingly extends over extensive parts or over the entire luminal surface 16.

The mural surface 12 and the luminal surface 16 are both covered by an SiC layer 18, 18′. Production of such SiC layers 18, 18′ is known in the art so that a detailed description need not be presented here.

The cavities 14 are filled with an antiproliferative substance (e.g., paclitaxel). A coating 20 containing attractors for endothelial cells is provided on the luminal side.

### EXAMPLES

Several exemplary embodiments are discussed below.

### Example 1

Rapamycin is dissolved with chloroform and extracted with beta-cyclodextrin in water.

### Example 2

Rapamycin is dissolved in chloroform together with a polylactide (for example, polylactides available under the brand names RESOMER™ L203 H or RESOMER™ L 203 S) and spray dried.

### Example 3

Production of a mixture of rapamycin with 6-O-palmitoyl-L-ascorbic acid or polyvinylpyrrolidone. The mixture may be introduced into the cavities in the form of a solution or as pressed tablets.

In another exemplary embodiment, the cavities are covered/coated with one or more substances from the group consisting of sugars, such as polysaccharides, glycans, glucose, glycogen, amylose, amylopectin, chitin, callose and cellulose, as well as fats, such as cholesterol, palm oil, partially hydrogenated soy oils and saturated oils.

All patents, patent applications and publications referred to herein are incorporated by reference in their entirety.

What is claimed is:

1. A stent having a base body of a bioinert metallic implant material, the stent comprising:
a) a base body at least partially covered with an SiC coating;
b) an external surface of the base body having a plurality of cavities which are at least partially filled with an antiproliferative active ingredient; and
c) a luminal surface of the base body having a coating comprising attractors for endothelial cells.

2. The stent of claim 1, wherein the luminal surface of the base body is contoured.

3. The stent of claim 1, wherein the base body comprises at least one material selected from the group consisting of CoCr, 316L and nitinol.

4. The stent of claim 1, wherein the antiproliferative active ingredient is selected from the group consisting of taxols and taxans.

5. The stent of claim 1, wherein the attractor for endothelial cells is selected from the group consisting of antibodies, aptamers, RGD peptides, and cyclic RGDs.

6. The stent of claim 1, wherein the antiproliferative active ingredient is selected from the group consisting of paclitaxel, sirolimus, derivatives of rapamycin, everolimus and biolomus.
